(19) United States
(12) Patent Application Publication
(10) Pub. No.: US 2012/0045788 A1
(43) Pub. Date: Feb. 23, 2012

(54) Device for sample preparation

(76) Inventors: Ashok K. Shukla, Ellicott City, MD (US); Mukta M. Shukla, Ellicott City, MD (US)

(21) Appl. No.: 13/199,104

(22) Filed: Aug. 19, 2011

Related U.S. Application Data
(60) Provisional application No. 61/401,928, filed on Aug. 20, 2010.

(51) Int. Cl. C12Q 1/02; G01N 33/68; C08F 10/00; C08F 14/26; C08B 3/06; C08F 112/08; C08G 75/23; C08F 10/06; C08F 10/02; C08B 1/00; B01J 8/00; C08F 212/10

(52) U.S. Cl. 435/29; 422/129; 422/240; 422/187; 422/69; 436/86; 521/143; 521/145; 521/147; 521/146; 521/180; 536/56; 536/69

(57) ABSTRACT

A pipette tip or tube containing chromatographic media contained and held in place in said tube by using low melting point porous polymer particles. Such pipette tips or tubes can be used for sample preparation, filtration and synthesis of small molecules and biomolecules.

Priority date claimed from provisional application No. 61/401,928, filed on Aug. 20, 2010.

### FIELD OF THE INVENTION

This invention relates to a device based on a pipette tip or tube that is designed to perform biological or chemical sample preparation, purification, separation or synthesis. The chromatographic media for sample preparation are fixed in the pipette tip. The embedding of the chromatographic media particles is achieved by using low melting point porous polyolefins or polymers. The melting point of the porous polyolefins or polymers is lower than the melting point of the polymer material from which the pipette tip is made. By using porous polyolefins or polymers, the volume reduction upon melting of the polyolefins or polymer allows for more chromatographic particles to be embedded in a pipette tip or tube for sample preparation.

The device described herein can be used for chemical or biochemical synthesis under drastic conditions such as high pH, low pH or highly polar or non-polar solvents. Furthermore, the device described herein can be used for the filtration, purification or separation of samples without the use of a filter since the chromatographic media are embedded in the polyolefins or polymers embedded in a pipette tip or tube. The device described herein can be applied for sample preparation using a vacuum, pressure, centrifugation, gravitation or other separation methods.

### BACKGROUND OF THE INVENTION

### Definitions of Key Terms

Tube: means any hollow encasing with one or more openings such as a capillary tube, pipette tip, a tube of any geometry with a cross section of any shape such as round, oval, square, cylindrical, triangular, elliptical, parabolic or any shape which can represent the cross section of a tube. Internal diameter of the tube is from 0.001-25 mm. The length of the tube can be from 0.1-10,000 mm.

Chromatographic media particles: includes regular, irregular, spherical or broken particles of silica, metal, polymers, carbon, metal oxides, cellulose, natural or synthetic polymers, fibers, and any other materials suitable for separating the components of a biological or chemical sample. These particles can be modified chemically, mechanically, physically or by affinity media. Furthermore, chromatographic media can be modified for a particular application such as peptide synthesis. These particles can be porous or nonporous. The pore size can be from 20-4,000 nm. The size of the particles can be from 0.001-1 mm.

Polyolefins or Polymers: includes but is not limited to materials such as PVC (polyvinylcloride), PE (polyethylene), PP (polypropylene) or any other inert synthetic or natural polymer. Mixtures of different polymers can also be used to decrease the melting point of polyolefins or polymers to be used for embedding materials.

Embedding of chromatographic media particles: means the chromatographic particles are embedded in the tube or pipette tip by using an inert polyolefin or polymer.

Samples: includes, but is not limited to, small molecules, proteins, peptides, synthetic molecules, DNA, RNA, lipids, carbohydrates and any other biomolecules. The sample may contain salts, detergents or any other impurities. The sample may also contain different types of mixtures of molecules which need to be purified.

Synthesis of molecules: means synthesis of molecules such as small molecules, proteins, peptides, DNA, RNA or nucleic acids. Synthesis also includes attachment of moieties such as affinity molecules or fluorescence molecules.

Although a spectrum of analytical methods for small sample separation and purification have been developed, a number of problems, such as the slow speed of the separation process and the loss of sample volumes, limit the quality of currently available methods.

In the present invention, we describe the use of a tube such as a pipette tip, which has embedded chromatographic particles. Currently, other devices are available, which use embedding of chromatographic media on the walls of pipette tips, or polymerization processes with the chromatographic particles, or monolithic material casting of media in the pipette tips.

The invention described herein has advantages over other available tube or pipette tip based devices for sample preparation:
1. In pipette tips in which the chromatographic material is embedded in the walls, if the material particles change their size, shape or structure during the sample preparation process involving the use of different solvents, the particles will detach from the wall of the tube or pipette tip. In addition, chromatographic particles such as fibers are difficult to attach or embed on the wall of a tube or pipette tip.
2. Methods which use a polymerization solution to cast chromatographic media particles inside a pipette tip can result in the particles becoming coated with the polymerization solution, reducing their chromatographic separation properties and effectiveness. In addition, under drastic conditions, the polymerized cast may dissolve, resulting in the release of the chromatographic particles from the pipette tip.
3. The use of monolithic casting limits the ability to use different chromatographic media in the same tip.
4. Liquid polymers used to cast chromatographic particles in a pipette tip typically solidify through exposure to UV light, heat or oxygen but can also result in coating of the chromatographic particles reducing their chromatographic separation properties and effectiveness.

To overcome the challenges described above, the present invention describes the use of low melting point porous polyolefins and polymers to fix chromatographic material particles in a pipette tip or tube. This method results in the chromatographic particles becoming embedded with the polyolefins or polymers, which become cast inside the pipette tip or tube. Furthermore, through controlled temperature heating, the porous property of the polyolefins or polymers can be retained. This allows for improved and more controlled flow of samples during sample preparation. In addition, the polyolefins or polymers described herein are inert to effects of drastic conditions such as low pH, high pH or organic or inorganic solvents. These drastic conditions are needed for processes such as peptide, protein, glycan or nucleic acid synthesis. Therefore, the invention described herein enables biomolecules synthesis in a pipette tip or tube under drastic conditions, which is not possible with other methods.

The invention described and claimed herein comprises a tube or pipette tip designed to perform filtration, sample separation, concentration and synthesis.

The various features of novelty, which characterize the invention, are pointed out with particularity in the claims annexed to and forming a part of this disclosure. For a better understanding of the invention, its advantages and objects, reference is made to the accompanying drawings and descriptive matter in which a preferred embodiment of the invention is illustrated.

### BRIEF DESCRIPTION OF THE DRAWINGS

The foregoing and still other objects of this invention will become apparent, along with various advantages and features of novelty residing in the present embodiments, from study of the following drawings, in which:

FIG. 1 is an expanded view of one embodiment of the pipette tip, according to the present invention, containing a chromatographic material and porous polymer particles within it.

FIG. 2 is an expanded view of one embodiment of the pipette tip, according to the present invention, containing two zones of chromatographic media.

FIG. 3 is an expanded view of one embodiment of a pipette tip, according to the present invention, containing loose chromatographic media on top of the casted solid comprised of porous polymer and chromatographic media.

### DESCRIPTION OF THE PREFERRED EMBODIMENTS

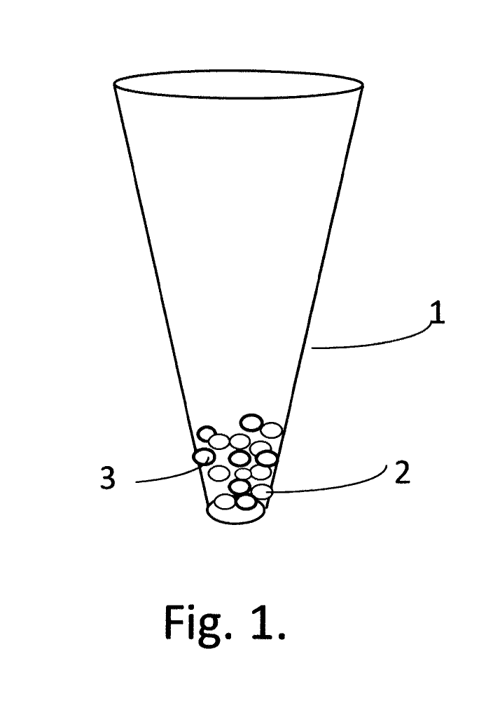

Referring to the drawings, FIG. 1 is an expanded view of one embodiment of the pipette tip or a tube (1), the chromatographic media particles (2) and the porous polyolefins or polymers (3). Said porous particles (3) are mixed with the chromatographic particles (2) in various proportions and placed in the tip as shown in FIG. 1. When this tip (1) is heated up to the melting point of the porous polymer, the chromatographic particles and porous particles fuse together in a cast. The volume of the porous particles (3) is reduced due to melting of the particles resulting in the formation of a porous structure with the chromatographic particles (2) embedded in it.

The melting point of the porous polymer particles can be reduced by mixing with additives which reduce the melting point of the polymer mixture. Furthermore, the melting temperature of the porous particles is adjusted in such a way that the particles just adhere to the chromatographic particles and maintain the porous properties of porous polymer particles. The application of pressure can also reduce the melting point of the porous polymer. This is useful for embedding temperature sensitive chromatographic particles (such as affinity media, immobilized enzymes or other temperature sensitive biomolecules).

Said pipette tip or tube (1) is made of one or more materials including but not limited to polymer, polypropylene, polyethylene, polyolefin, polytetrafluoroethylene, polysulfone, polyethersulfone, cellulose acetate, polystyrene, polystyrene/acrylonitrile copolymer, PVDF, metal and glass. Said pipette tip or tube (1) can have a volume anywhere from 0.0001 to 100 milliliters.

The internal diameter of said pipette tip or tube can be from 0.001-25 mm. Said pipette tip or tube (1) can be any type of holding unit such as a tube, housing, column, vial or any other type of holding unit suited to the sample preparation process. Said pipette tip or tube (1) can be of any shape or size and can be straight, tapered or any other geometry.

The particles of the chromatographic media (2) described in the present invention are any types of particles of any type of separation media suited for the separation, preparation, analysis, filtration, purification and synthesis of samples. For example, said separation media can be a chromatographic material selected from the group comprised of, but not limited to, porous chromatography materials; non-porous chromatography materials; silica materials; non-silica materials; polymer-based materials; natural polymer based, active charcoal; zirconium oxide; titanium oxide; polystyrene; carbon; cellulose, modified cellulose, anion exchanger, cation exchanger, affinity chromatography materials; polymers; gels; bacteria; living cells; solid powders; and combinations thereof. The chromatographic particles (2) are of any size between 0.1-10,000 micrometers. The pore size in the particle is between 20-10,000 Angstrom.

Said porous polymer (3) is made of one or more materials including but not limited to polymer, polypropylene, polyethylene, polyolefin, polytetrafluoroethylene, polysulfone, polyethersulfone, cellulose, cellulose acetate, polystyrene, polystyrene/acrylonitrile copolymer, PVDF. Any additive can be added to lower the melting point of porous particles. The porous particles (3) are of any size between 0.1-10,000 micrometers. The pour size is between 20-10,000 Angstrom.

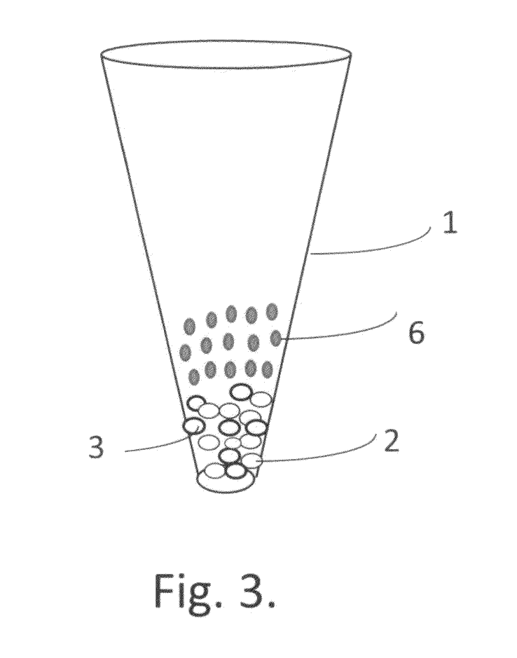

Furthermore, said tube (1) can be of any form selected from the group comprised of a hollow interior form or a partially hollow interior form. Said partially hollow interior form can consist of said tube (1) filled with a separation medium such that particles of said medium (6), FIG. 3 are packed in said tube (1) while still permitting the passage of the sample into or through said tube (1).

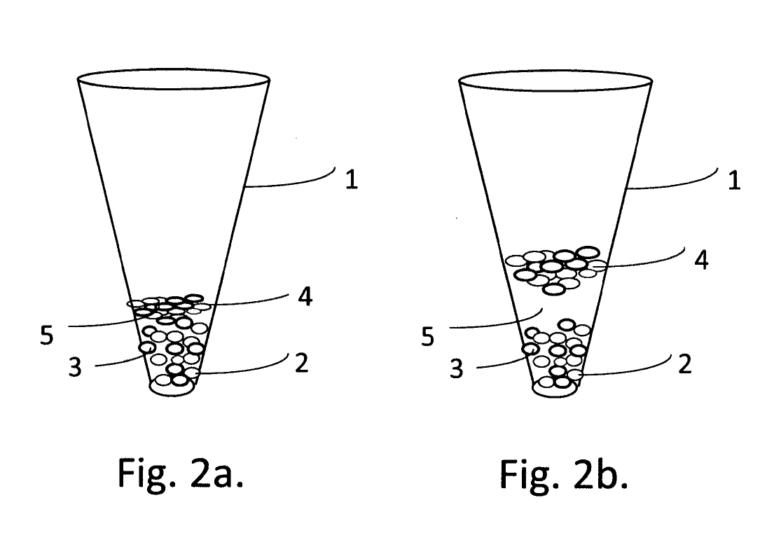

FIG. 2a shows two or more zones of different or same chromatographic media (2) with different or the same melted porous polymers (3) can be created in the same pipette tip or tube (1). These zones (4) can be separated by an empty space (5), as shown in FIG. 2b, or can be created without the empty space as shown in FIG. 2a. The empty space (5) can be created by adding a layer of some soluble particles, such as salt, between the two zones. Once the zones have been created by melting the polymer mixture, the empty space is created by washing out or dissolving the soluble particles. Such multi zones can enable more selective purification of samples.

Furthermore, the particles of the chromatographic or separation media (2) can be chemically, physically or biologically modified. Said particles (2) can be of any shape or size such as a size ranging from micrometers to millimeters in each dimension. In addition, the particles of said separation media (2) can be of a shape selected from the group comprised of, but not limited to spherical shapes, cubical shapes, cylindrical shapes, oval shapes, irregular shapes, fiber and combinations thereof.

The device, according to the present invention, can be present in any multi-format form such that said device exists as part of a multi-tube or multi-column format selected from the group comprised of 8-, 12-, 24-, 48-, 96-, 384-, 1536- or higher tube or column formats.

Furthermore, the present invention can be used for any sample preparation methods wherein said sample preparation methods are selected from the group comprised of chromatography; high pressure liquid chromatography (HPLC); electrophoresis; gel filtration; sample centrifugation; on-line sample preparation; diagnostic kits testing; diagnostic testing; transport of chemicals; transport of biomolecules; high throughput screening; affinity binding assays; purification of said sample; size-based separation of the components of said sample; physical properties based separation of the components of said sample; chemical properties based separation of the components of said sample; biological properties based separation of the components of said sample; electrostatic properties based separation of the components of said sample; biomolecules synthesis; and, combinations thereof. Also, the device of the present invention can be part of a larger device or can have a unique function such as being a reaction chamber or spin column.

This invention is useful for the synthesis of peptides and other biomolecules, because the use of suitable porous polymers and chromatographic particles can make the device described by the present invention very inert, enabling sample preparation, such as biomolecules synthesis, in drastic conditions.

Furthermore, the present invention can be used for the preparation of any types of sample where said samples are selected from the group comprised of biological samples; protein containing samples; nucleic acid containing samples; lipid containing samples; carbohydrates containing samples; cell containing samples; blood containing samples; tissue containing samples; living matter containing samples; mucus containing samples; serum containing samples; chemical samples; biochemical samples; radioactive samples; and, combinations thereof.

FIG. 3 is an expanded view of one embodiment of the pipette tip (1), according to the present invention, containing a chromatography material (2) within it. FIG. 3 also shows a loose chromatographic material (6) which is placed on top of the chromatographic material (2) embedded in the polymer (3). Said chromatography media (2 and 6) can consist of one or more different types of chromatography or separation materials including, but not limited to, chromatographic silica, affinity, IMAC, polystyrene, carbon, polymers, media, gels, bacteria, living cells, solid powders or any other media used for the purposes of sample filtration, separation or purification. The chromatographic materials (2 and 6) can also be composed of non-silica, polymer-based, active charcoal, zirconium, titanium, metal oxide or other materials. In this case the cast chromatographic media (2) serves as a filter to hold the loose chromatographic media particles within the tip (6).

The chromatographic media (2 and 6) can also be a mixture of materials with different particle sizes or different types of materials such as cation and anion exchange materials, affinity materials, and normal phase or reverse phase materials. Said chromatography or separation material (2 and 6) can be in any shape or form including but not limited to particle form, powder form, or in any other physical configuration suited to the design of the pipette tip and the experimental conditions. Furthermore, the particles of said chromatographic or separation material (2 and 6) can be chemically or physically modified and may be porous or non-porous. The sizes of the inert or chromatographic material particles can be from nanometers to micrometers.

The passage of the sample through the pipette tip (1) can be performed using any relevant methods, including but not limited to, centrifugation, gravitation, vacuum suction, pressure application or syringe-based sample delivery through the container.

Said tube (1) can be arranged in multiple units of the pipette tip or tube (1), joined together in a 96-tube format. Multiple units of said pipette tips (1) can be joined together to develop a system for the simultaneous preparation of multiple samples. Said pipette tips (1) can be joined together in any type of configuration including but not limited to 2-unit, 8-unit, 48-unit, 96-unit, 384-unit or 1536-unit formats. The pipette tip (1) is either a single unit or a multi-unit system and can be combined with a piston or similar device designed to pull the sample into the tip or push the sample out of the tip.

The pipette tip or tube described in the present invention has many sample preparation, filtration, purification and synthesis applications including but not limited to the purification of DNA, proteins, peptides, lipids, carbohydrates, vitamins and other chemicals and biomolecules. Separation of sample components can be based on size, chemical properties or physical properties of the sample's component molecules and particles. Samples purified by these methods can be used for further analysis, through mass spectrometry, High Performance Liquid Chromatography (HPLC), electrophoresis, capillary electrophoresis, NMR, enzyme assays, protein binding assays and other chemical or biochemical reactions. The invention described herein is suited for the synthesis of peptides, DNA, RNA, nucleic acids, proteins, lipids, carbohydrates and glycans.

The broader usefulness of the invention may be illustrated by the following examples.

### Example 1

### Use of the Present Invention for Peptide Sample Preparation

In this experiment, we used a 1-10 microliter micro pipette tip and placed a mixture of C18 chromatographic particles (size 10 micrometers) and polyethylene porous particles (size 30 micrometers) in a 1:1 ratio in the tip. The height of the mixture of particles is 2 mm in the narrow end of the tip. The tip containing the particle mixture was heated for 10 min. at 110 degrees C. A 10 microliter peptide solution containing buffer was pipetted in and out of the pipette tip by using a micro pipettor. The peptide was retained in the tip while water and impurities passed through the lower, narrow opening. The tip was washed with 20 microliters of water a few times and the peptide was then eluted from the tip using 10 microliters of 50 percent isopropanol. The purified peptide sample was then analyzed by HPLC.

### Example 2

### Peptide Synthesis

In this experiment, we used a 1-10 microliter micro pipette tip and placed a mixture of C18 chromatographic particles (size 10 micrometers) and cellulose fiber particles in a 1:1 ratio in the tip. The cellulose containing tip was made by the method described in Example 1 above. The OH groups of cellulose were attached to activated glycine as described in the literature for cellulose spot synthesis (Frank R. (1992), Spot-synthesis: An easy technique for the positionally addressable, parallel chemical synthesis on a membrane support, Tetrahedron, 48, 9217-9232). In place of a cellulose sheet, as described in the publication, all the reactions described herein under Example 2, were carried out in a cellulose embedded pipette tip. The peptides synthesized by the tips described in the present invention, were further analyzed by HPLC and MS. Similarly, other activated chromatographic media can be used for peptide, DNA, RNA and glycan synthesis. The use of a pipette tip or tube for biomolecules synthesis, as described in the present invention, enables easier and more efficient peptide synthesis and also enables high throughput through automation of peptide synthesis using the device described in the present invention. This is a significant advantage over existing methods.

While a specific embodiment of the invention has been shown and described in detail to illustrate the application of the principles of the invention, it is understood that the invention may be embodied otherwise without departing from such principles and that various modifications, alternate constructions, and equivalents will occur to those skilled in the area given the benefit of this disclosure and the embodiment described herein, as defined by the appended claims.

What is claimed is:

1. A tube for sample preparation where said tube contains chromatographic media mixed with porous polymer particles to form a mixture and said mixture is fixed in place within said tube by the application of heat or pressure or a combination of both.

2. The porous polymer particles of claim 1, wherein the melting point of said particles is lower than the melting point of the material comprising said tube of claim 1.

3. The tube of claim 1, wherein said tube is of a shape selected from the group consisting of a cylindrical shape; a spherical shape; an elongated shape with a square cross-section; an elongated shape with a rectangular cross-section; an elongated shape with a polygonal cross-section; an elongated shape with an oval cross-section; an elongated shape with an irregularly shaped cross-section; an elongated shape with varying cross-sections along the length of said tube; and, combinations thereof.

4. The tube of claim 1, wherein the ends of said tube are selected from the group consisting of an open top end and an open bottom end; an open top end and a closed bottom end; a closed top end and an open bottom end; a closed top end and a closed bottom end; a tapered open end; a tapered closed end; and, combinations thereof.

5. The tube of claim 1, wherein said tube is selected from the group comprised of a capillary tube; a pipette tip; a chromatography column; a spin column and, combinations thereof.

6. The tube of claim 1, wherein said tube is made of a material selected from the group consisting of porous materials, non-porous materials, polyethylene, polypropylene, polyolefins, polytetrafluoroethylene, polysulfone, polyethersulfone, cellulose acetate, polystyrene, polystyrene/acrylonitrile copolymer, PVDF, glass, metal, silica, paper, heat shrink materials, and combinations thereof.

7. The chromatographic media of claim 1, wherein said media is a chromatography material selected from the group consisting of porous chromatography materials; non-porous chromatography materials; silica materials; non-silica materials; polymer-based materials; active charcoal; metal oxides; zirconiumdioxide; titaniumdioxide; polystyrene; carbon; cellulose; affinity chromatography materials; natural polymers; synthetic polymers; gels; bacteria; cells; solid powders; fibers; and combinations thereof.

8. The chromatographic media of claim 1, wherein the particles of said media are chemically, physically or biologically modified.

9. The Chromatographic media of claim 1, wherein the particles of said media are of a size ranging from micrometers to millimeters in each dimension.

10. The chromatographic media of claim 1, wherein the particles of said media are of a shape selected from the group consisting of spherical shapes, cubical shapes, cylindrical shapes, oval shapes, irregular shapes, fiber and combinations thereof.

11. The tube of claim 1, wherein said tube contains more than one chromatographic media zone.

12. The tube of claim 1, wherein, said tube contains more than one chromatographic media zone and said zones are separated by empty space.

13. The porous polymer particles of claim 1, wherein said particles are selected from the group consisting of porous chromatography materials; polymer; polypropylene; polyethylene; polyolefin; polytetrafluoroethylene; polysulfone; polyethersulfone; cellulose, cellulose acetate, polystyrene, polystyrene/acrylonitrile copolymer, and combinations thereof.

14. The porous polymer particles of claim 1, wherein said low melting porous polymer particles are mixed with other particles to lower the melting point.

15. The chromatographic media as in claim 1, wherein said chromatographic or separation material can be one type of material or a mixture of different sizes of particles or different types of materials.

16. The tube of claim 1, wherein said tube exists as part of a multi-tube or multi-column format selected from the group comprised of 8-, 12-, 24-, 48-, 96-, 384-, 1536- or higher tube or column formats.

17. A tube for synthesis of biomolecules where said tube contains chromatographic media mixed with porous polymer particles to form a mixture and said mixture is fixed in place within said tube by the application of heat or pressure or a combination of both.

18. The tube of claim 17, wherein said synthesis selected from the group consisting of synthesis of biomolecules, peptides, DNA, RNA, nucleic acids, proteins, lipids, carbohydrates, and glycans.
